(19) United States
(12) Patent Application Publication
(10) Pub. No.: US 2001/0016330 A1
(43) Pub. Date: Aug. 23, 2001

(54) Apparatus and methods for identifying quenching effects in luminescence assays

(75) Inventors: John C. Owicki, Palo Alto, CA (US); Douglas N. Modlin, Palo Alto, CA (US); Todd E. French, Cupertino, CA (US)

Correspondence Address: James R. Abney, KOLISCH, HARTWELL, DICKINSON,, McCORMACK & HEUSER, 520 S.W. Yamhill Street, Suite 200, Portland, OR, 97204, US

(73) Assignee: LJL BioSystems, Inc.

(21) Appl. No.: 09/766,131

(22) Filed: Jan. 19, 2001

Related U.S. Application Data
(63) Continuation of application No. PCT/US99/16286, filed on Jul. 26, 1999.
(60) Provisional application No. 60/094,306, filed on Jul. 27, 1998.

(51) Int. Cl. G01N 33/53; G01N 33/542

(52) U.S. Cl. 435/7.71; 435/7.9

(57) ABSTRACT

Apparatus and methods for identifying and correcting for quenching in luminescence assays using luminescence lifetimes and/or luminescence intensities. One aspect of the invention involves identifying quenching using combinations of luminescence lifetimes and/or intensities. Another aspect of the invention involves correcting for quenching by eliminating false positives or false negatives due to quenching in luminescence assays.

### CROSS-REFERENCES TO RELATED APPLICATIONS

This application is a continuation of PCT Patent Application Ser. No. PCT/US99/16286, filed Jul. 26, 1999, which is incorporated herein by reference.

This application claims priority from U.S. Provisional Patent Application Ser. No. 60/094,306, filed Jul. 27, 1998, which is incorporated herein by reference.

This application incorporates by reference the following U.S. patent application Ser. No. 09/156,318, filed Sep. 18, 1998; and Ser. No. 09/349,733, filed Jul. 8, 1999.

This application also incorporates by reference the following PCT patent application Ser. No. PCT/US98/23095, filed Oct. 30, 1998; Ser. No. PCT/US99/01656, filed Jan. 25, 1999; Ser. No. PCT/US99/03678, filed Feb. 19, 1999; Ser. No. PCT/US99/08410, filed Apr. 16, 1999; Ser. No. PCT/US99/16057, filed Jul. 15, 1999; Ser. No. PCT/US99/16453, filed Jul. 21, 1999; Ser. No. PCT/US99/16621, filed Jul. 23, 1999; and Ser. No. PCT/US99/16287, filed Jul. 26, 1999.

This application also incorporates by reference the following U.S. provisional patent application Ser. No. 60/100,817, filed Sep. 18, 1998; Ser. No. 60/100,951, filed Sep. 18, 1998; Ser. No. 60/104,964, filed Oct. 20, 1998; Ser. No. 60/114,209, filed Dec. 29, 1998; Ser. No. 60/116,113, filed Jan. 15, 1999; Ser. No. 60/117,278, filed Jan. 26, 1999; Ser. No. 60/119,884, filed Feb. 12, 1999; Ser. No. 60/121,229, filed Feb. 23, 1999; Ser. No. 60/124,686, filed Mar. 16, 1999; Ser. No. 60/125,346, filed Mar. 19, 1999; Ser. No. 60/126,661, filed Mar. 29, 1999; Ser. No. 60/130,149, filed Apr. 20, 1999; Ser. No. 60/132,262, filed May 3, 1999; Ser. No. 60/132,263, filed May 3, 1999; Ser. No. 60/135,284, filed May 21, 1999; Ser. No. 60/138,311, filed Jun. 9, 1999; Ser. No. 60/138,438, filed Jun. 10, 1999; Ser. No. 60/138,737, filed Jun. 11, 1999; Ser. No. 60/138,893, filed Jun. 11, 1999; and Ser. No. 60/142,721, filed Jul. 7, 1999.

This application also incorporates by reference the following publications: Max Born and Emil Wolf, Principles of Optics (6^{th} ed. 1980); Richard P. Haugland, Handbook of Fluorescent Probes and Research Chemicals (6^{th} ed. 1996); and Joseph R. Lakowicz, Principles of Fluorescence Spectroscopy (1983).

### FIELD OF THE INVENTION

The invention relates to luminescence assays. More particularly, the invention relates to apparatus and methods for identifying and correcting for quenching in luminescence assays using luminescence lifetimes and/or intensities.

### BACKGROUND OF THE INVENTION

Luminescence is the emission of light from excited electronic states of atoms or molecules. Luminescence generally refers to all kinds of light emission, except incandescence, and may include photoluminescence, chemiluminescence, and electrochemiluminescence, among others. In photoluminescence, including fluorescence and phosphorescence, the excited electronic state is created by the absorption of electromagnetic radiation. In chemiluminescence, which includes bioluminescence, the excited electronic state is created by a transfer of chemical energy. In electrochemiluminescence, the excited electronic state is created by an electrochemical process.

Luminescence assays are assays that use luminescence emissions from luminescent analytes to study the properties and environment of the analyte, as well as binding reactions and enzymatic activities involving the analyte, among others. In this sense, the analyte may act as a reporter to provide information about another material or target to substance that is the true focus of the assay. Luminescence assays may use various aspects of the luminescence, including its intensity, polarization, lifetime, and sensitivity to energy transfer, among others. Luminescence assays also may use time-independent (steady-state) and/or time-dependent (time-resolved) properties of the luminescence.

Unfortunately, luminescence assays are subject to artifacts that alter the apparent luminescence and luminescence properties of the analyte and thus the accuracy, repeatability, and reliability of the assay. Some artifacts increase the apparent luminescence of the analyte, causing intensity-based assays to overreport the amount of light emitted by the analyte. Such artifacts include background. Other artifacts decrease the apparent luminescence of the analyte, causing intensity-based assays to underreport the amount of light emitted by the analyte. Such artifacts include quenching.

Generally, quenching refers to any process that decreases the luminescence intensity of a given substance. Quenching can arise from a variety of processes, including excited state reactions, energy transfer, collisions, and complex formation. Dynamic (or collisional) quenching results from collisional encounters between a luminophore and a dynamic quencher. For this reason, dynamic quenching may be more significant for long-lifetime luminophores, because luminophore and quencher may diffuse significantly during the long lifetime and so be more likely to interact collisionally. Static quenching results from complex formation between a luminophore and a static quencher. Both static and dynamic quenching require molecular contact between the luminophore and quencher. In the case of dynamic quenching, the quencher and luminophore must diffuse together during the lifetime of the excited state. Upon contact, the luminophore returns to the ground state, without emission of a photon. In the case of static quenching, a complex is formed between the luminophore and the quencher, and this complex is nonfluorescent. In either event, the luminophore and quencher must be in contact.

More specifically, quenching may be defined in the context of a given assay as any process that decreases the luminescence intensity of a given substance, other than a process of interest. For example, as described below, in assays known as resonance energy transfer assays, interactions between assay species may lead to a decrease in the luminescence of one of the species. In such an assay, the energy transfer leading to such a decrease would be excluded from the definition of quenching.

A variety of compounds can act as quenchers; examples are described in Joseph R. Lakowicz, Principles of Fluorescence Spectroscopy (1983), which is incorporated herein by reference. Probably the best known quencher is molecular oxygen, which quenches almost all known luminophores. Generally, whether a particular compound will act as a quencher depends on the mechanisms by which it can interact with each particular luminophore, which in turn depends on the relative structures of the compound and luminophore in a given environment.

Apparent quenching also can occur, due to optical properties of the sample. For example, high optical densities or turbidity can decrease luminescence intensities. This Who type of quenching contains little molecular information.

Luminescence assays form the foundation of many assays employed in screening libraries of compounds to provide leads for the development of new therapeutic drugs. (See, for example, the patent applications incorporated by reference above.) In such screening, hundreds of thousands of samples may be analyzed each day, and during primary screening typically only about 0.1% of the samples will give positive results (“hits”) that merit further investigation. Unfortunately, the number of “hits” may be significantly overestimated if mechanisms other than those underlying the assay lead to changes in luminescence or luminescence properties. For example, if only 1% of the compounds caused quenching that might be confused with a hit, then the number of false positives caused by quenching would outnumber the number of true hits by a factor of 10. If a significant portion of false positive tests due to quenching could be identified and treated as negative hits, or retested under different conditions, then the efficiency of the screening protocol could be improved dramatically, resulting in a savings of time and money.

### SUMMARY OF THE INVENTION

The invention provides apparatus and methods for identifying and correcting for quenching in luminescence assays using combinations of luminescence lifetimes and/or luminescence intensities.

### BRIEF DESCRIPTION OF THE DRAWINGS

FIG. 1 is a schematic view of luminescently labeled molecules, showing how molecular reorientation affects luminescence polarization.

FIG. 2 is a schematic view of a frequency-domain time-resolved measurement, showing the definitions of phase angle (phase) φ and demodulation factor (modulation) M.

FIG. 3 is a graph of donor and acceptor intensity versus time in a time-resolved resonance energy transfer assay, showing how time and energy transfer affect intensity.

FIG. 4 is a graph of donor and acceptor intensity versus time in a time-resolved resonance energy transfer assay, showing how time, energy transfer, and static quenching affect intensity.

FIG. 5 is a graph of donor and acceptor intensity versus time in a time-resolved resonance energy transfer assay, showing how time, energy transfer, and dynamic quenching affect intensity.

FIG. 6 is a schematic view of an apparatus for detecting light in accordance with the invention.

FIG. 7 is a schematic view of an alternative apparatus for detecting light in accordance with the invention.

FIG. 8 is a partially schematic perspective view of the apparatus of FIG. 7.

FIG. 9 is a schematic view of photoluminescence optical components from the apparatus of FIG. 7.

FIG. 10 is a schematic view of chemiluminescence optical components from the apparatus of FIG. 7.

FIG. 11 is a partially exploded perspective view of a housing for the apparatus of FIG. 7.

FIG. 12 is a schematic view of an alternative apparatus for detecting light in accordance with the invention.

### DETAILED DESCRIPTION OF THE INVENTION

The invention provides apparatus and methods for identifying and correcting for quenching in luminescence assays, including time-resolved resonance energy transfer (RET) assays. One aspect of the invention involves identifying quenching using combinations of luminescence lifetimes and/or intensities. Another aspect of the invention involves correcting for quenching by eliminating false positives or false negatives due to quenching in luminescence assays. These and other aspects of the invention are described in the following three sections: (1) luminescence assays, (2) application of methods, and (3) description of apparatus.

### 1. Luminescence Assays

Luminescence is the emission of light from excited electronic states of atoms or molecules. As described below, luminescence may be used in a variety of assays, including (A) intensity assays, (B) energy transfer assays, (C) polarization assays, (D) time-resolved assays, and (E) miscellaneous assays.

A. Intensity Assays

Luminescence intensity assays involve monitoring the intensity (or amount) of so light emitted from a composition. The intensity of emitted light will depend on the extinction coefficient, quantum yield, and number of the luminescent analytes in the composition, among others. These quantities, in turn, will depend on the environment of the analyte, among others, including the proximity and efficacy of quenchers and energy transfer partners. Thus, luminescence intensity assays may be used to study binding reactions, among other applications.

B. Energy Transfer Assays

Energy transfer is the transfer of luminescence energy from a donor luminophore to an acceptor without emission by the donor. In energy transfer assays, a donor luminophore is excited from a ground state into an excited state by absorption of a photon. If the donor luminophore is sufficiently close to an acceptor, excited-state energy may be transferred from the donor to the acceptor, causing donor luminescence to decrease and acceptor luminescence to increase (if the acceptor is luminescent). The efficiency of this transfer is very sensitive to the separation R between donor and acceptor, decaying as I/R⁻⁶. Energy transfer assays use energy transfer to monitor the proximity of donor and acceptor.

Some energy transfer assays focus on an increase in energy transfer as donor and acceptor are brought into proximity. These assays may be used to monitor binding, as between two molecules X and Y to form a complex X:Y. Here, colon (:) represents a noncovalent interaction. In these assays, one molecule is labeled with a donor D, and the other molecule is labeled with an acceptor A, such that the interaction between X and Y is not altered appreciably. Independently, D and A may be covalently attached to X and Y, or covalently attached to binding partners of X and Y.

Other energy transfer assays focus on a decrease in energy transfer as donor and acceptor are separated. These assays may be used to monitor cleavage, as by hydrolytic digestion of doubly labeled substrates (peptides, nucleic acids). In a typical application, two ends of a polypeptide might be labeled with D and A, so that cleavage of the polypeptide by an endopeptidase will separate D and A and thereby reduce energy transfer.

Energy transfer between D and A may be monitored in various ways. For example, energy transfer may be monitored by observing an energy-transfer induced decrease in the emission intensity of D and increase in the emission intensity of A (if A is a luminophore). Energy transfer also may be monitored by observing an energy-transfer induced decrease in the lifetime of D and increase in the apparent lifetime of A.

In a preferred mode, a long-lifetime luminophore is used as a donor, and a short-lifetime luminophore is used as an acceptor. Suitable long-lifetime luminophores include metal-ligand complexes containing ruthenium, osmium, etc., and lanthanide chelates containing europium, terbium, etc. In time-domain assays, the donor is excited using a flash of light having a wavelength near the excitation maximum of D. Next, there is a brief wait, so that electronic transients and/or short-lifetime background luminescence can decay. Finally, donor and/or acceptor luminescence intensity is detected and integrated. In frequency-domain assays, the donor is excited using time-modulated light, and the phase and/or modulation of the donor and/or acceptor emission is monitored relative to the phase and/or modulation of the excitation light. In both assays, donor luminescence is reduced if there is energy transfer, and acceptor luminescence is observed only if there is energy transfer.

C. Polarization Assays

Luminescence polarization assays involve the absorption and emission of polarized light, and typically are used to study molecular rotation. (Polarization describes the direction of light's electric field, which generally is perpendicular to the direction of light's propagation.)

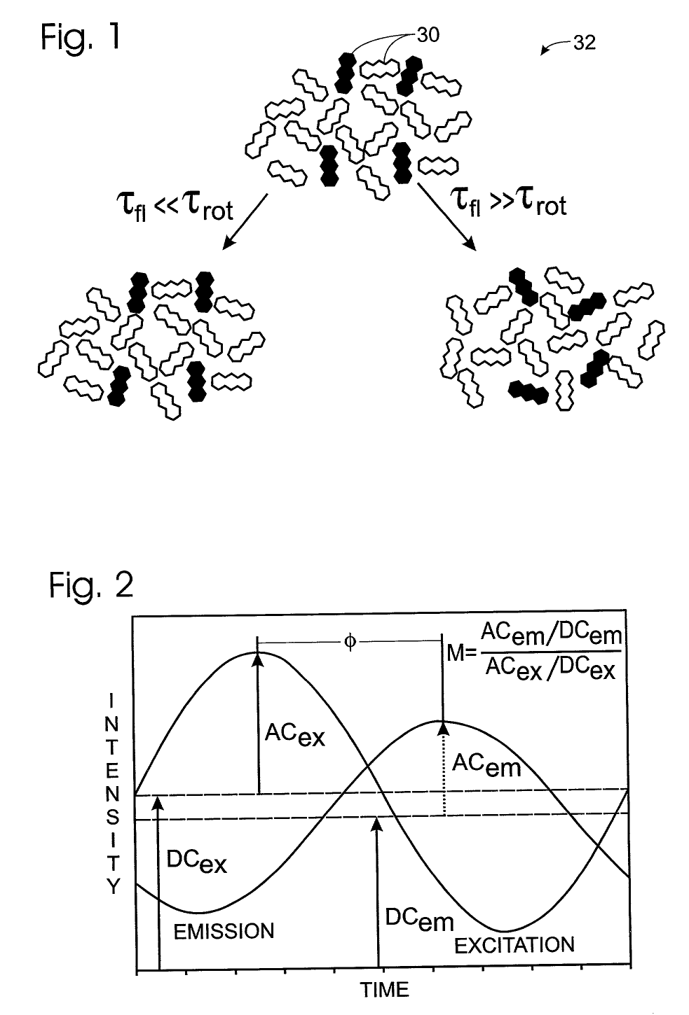

FIG. 1 is a schematic view showing how luminescence polarization is affected by molecular rotation. In a luminescence polarization assay, specific molecules 30 within a composition 32 are labeled with one or more luminophores. The composition then is illuminated with polarized excitation light, which preferentially excites luminophores having absorption dipoles aligned parallel to the polarization of the excitation light. These molecules subsequently decay by preferentially emitting light polarized parallel to their emission dipoles. The extent to which the total emitted light is polarized depends on the extent of molecular reorientation during the time interval between luminescence excitation and emission, which is termed the luminescence lifetime, τ. The extent of molecular reorientation in turn depends on the luminescence lifetime and the size, shape, and environment of the reorienting molecule. Thus, luminescence polarization assays may be used to quantify binding reactions and enzymatic activity, among other applications. In particular, molecules commonly rotate via diffusion with a rotational correlation time τᵣₒₜ that is proportional to their size. Thus, during their luminescence lifetime, relatively large molecules will not reorient significantly, so that their total luminescence will be relatively polarized. In contrast, during the same time interval, relatively small molecules will reorient significantly, so that their total luminescence will be relatively unpolarized.

The relationship between polarization and intensity is expressed by the following equation:

$$P = \frac{{I}_{∥} - {I}_{⊥}}{{I}_{∥} + {I}_{⊥}} \qquad ( 1 )$$

Here, P is the polarization, I_{∥} is the intensity of luminescence polarized parallel to the polarization of the excitation light, and I_{⊥} is the intensity of luminescence polarized perpendicular to the polarization of the excitation light. P generally varies from zero to one-half for randomly oriented molecules (and zero to one for aligned molecules). If there is little rotation between excitation and emission, I_{∥} will be relatively large, I_{⊥} will be relatively small, and P will be close to one-half. (P may be less than one-half even if there is no rotation; for example, P will be less than one if the absorption and emission dipoles are not parallel.) In contrast, if there is significant rotation between absorption and emission, I_{∥} will be comparable to I_, and P will be close to zero. Polarization often is reported in milli-P (mP) units (1000×P), which for randomly oriented molecules will range between 0 and 500, because P will range between zero and one-half.

Polarization also may be described using other equivalent quantities, such as anisotropy. The relationship between anisotropy and intensity is expressed by the following equation:

$$r = \frac{{I}_{∥} - {I}_{⊥}}{{I}_{∥} + 2 {I}_{⊥}} \qquad ( 2 )$$

Here, r is the anisotropy. Polarization and anisotropy include the same information, is although anisotropy may be more simply expressed for systems containing more than one luminophore. In the description and claims that follow, these terms may be used interchangeably, and a generic reference to one should be understood to imply a generic reference to the other.

The relationship between polarization, luminescence lifetime, and rotational correlation time is expressed by the Perrin equation:

$$( \frac{1}{P} - \frac{1}{3} ) = ( \frac{1}{{P}_{0}} - \frac{1}{3} ) \cdot ( 1 + \frac{τ}{{τ}_{rot}} ) \qquad ( 3 )$$

Here, P₀ is the polarization in the absence of molecular motion (intrinsic polarization), τ is the luminescence lifetime (inverse decay rate) as described above, and τᵣₒₜ is the rotational correlation time (inverse rotational rate) as described above.

The Perrin equation shows that luminescence polarization assays are most sensitive when the luminescence lifetime and the rotational correlation time are similar. Rotational correlation time is proportional to molecular weight, increasing by about 1 nanosecond for each 2,400 Dalton increase in molecular weight (for a spherical molecule). For shorter lifetime luminophores, such as fluorescein, which has a luminescence lifetime of roughly 4 nanoseconds, luminescence polarization assays are most sensitive for molecular weights less than about 40,000 Daltons. For longer lifetime probes, such as Ru(bpy)₂dcbpy (ruthenium 2,2′-dibipyridyl 4,4′-dicarboxyl-2,2′-bipyridine), which has a lifetime of roughly 400 nanoseconds, luminescence polarization assays are most sensitive for molecular weights between about 70,000 Daltons and 4,000,000 Daltons.

D. Time-Resolved Assays

Time-resolved assays involve measuring the time course of luminescence emission. Time-resolved assays may be conducted in the time domain or in the frequency domain, both of which are functionally equivalent. In a time-domain measurement, the time course of luminescence is monitored directly. Typically, a composition containing a luminescent analyte is illuminated using a narrow pulse of light, and the time dependence of the intensity of the resulting luminescence emission is observed, although other protocols also may be used. For a simple molecule, the luminescence commonly follows a single-exponential decay.

In a frequency-domain measurement, the time course of luminescence is monitored indirectly, in frequency space. Typically, the composition is illuminated using light whose intensity is modulated sinusoidally at a single modulation frequency f; although other protocols (such as transforming time-domain data into the frequency domain) also may be used. The intensity of the resulting luminescence emission is modulated at the same frequency as the excitation light. However, the emission will lag the excitation by a phase angle (phase) φ, and the intensity of the emission will be demodulated relative to the intensity of the excitation by a demodulation factor (modulation) M.

FIG. 2 shows the relationship between emission and excitation in a single-frequency frequency-domain experiment. The phase φ is the phase difference between the excitation and emission. The modulation M is the ratio of the AC amplitude to the DC offset for the emission, relative to the ratio of the AC amplitude to the DC offset for the excitation. The phase and modulation are related to the luminescence lifetime τ by the following equations:

ωτ=tan (φ) (4)

$$ω τ = \sqrt{\frac{1}{{M}^{2}} - 1} \qquad ( 5 )$$

Here ω is the angular modulation frequency, which equals 2π times the modulation frequency. For maximum sensitivity, the angular modulation frequency should be roughly the inverse of the luminescence lifetime. Lifetimes of interest in high-throughput screening vary from less than 1 nanosecond to greater than 1 milliseconds. Therefore, instruments for high-throughput screening should be able to cover modulation frequencies from about 200 Hz to about 200 MHz.

E. Miscellaneous Assays

Additional luminescence assays, including total internal reflection fluorescence (TIR), fluorescence correlation spectroscopy (FCS), and fluorescence recovery after So photobleaching (FRAP), as well as their phosphorescence analogs, may be conducted using procedures outlined in the patent applications and books cross-referenced above and/or generally known to persons of ordinary skill in the art.

### 2. Description of Methods

The invention provides methods for identifying and correcting for interference from luminescence quenching in luminescence assays. These methods may include performing a luminescence assay, and comparing measured and expected assay results using combinations of luminescence lifetimes and/or luminescence intensities to identify and correct for false hits due to quenching.

The invention may be applied to a variety of luminescence assays, particularly assays involving measurement of luminescence intensities. This section presents an application of the invention to resonance energy transfer (RET) assays.

In a typical RET assay, donors are excited from their ground states into an excited state by the absorption of a photon, and the proximity of acceptors is monitored using the effects of energy transfer on donor and/or acceptor lifetimes and/or intensities. Unfortunately, RET assays may be complicated by mechanisms that alter donor and acceptor properties in ways that mimic or mask the effects of energy transfer. The invention provides apparatus and methods for identifying and correcting for these mechanisms, particularly quenching.

The methods provided by the invention may include labeling a first binding partner with an energy-transfer donor, D, and labeling a second binding partner with an energy-transfer acceptor, A, as described above. The first and second binding partners may be free or bound together, so that donor and acceptor bound to these partners may be found as four species: free donor (D_{f}), bound donor (D_{b}), free acceptor (A_{f}), and bound acceptor (A_{b}).

Generally, each of D_{f}, D_{b}, A_{f}, and A_{b} may be characterized by different spectroscopic properties, including lifetimes and excitation/emission spectra. In particular, D_{b} and A_{b} may have different spectroscopic properties than D_{f} and A_{f}, respectively, due to energy transfer and differences in quenching efficiency. Typically, D and A are chosen such that the lifetime of D_{f} is (substantially) longer than the lifetime of A_{f}, although RET assays do not require such a choice.

The rate of decay, P(t), of an excited luminophore can be described using a first-order differential equation:

$$\frac{ⅆ P ( t )}{ⅆ t} = - kP ( t ) \qquad ( 6 )$$

Here, k is a rate constant that includes contributions from photon production (e.g., fluorescence and phosphorescence), energy transfer, quenching, and other processes. The solution of Equation 6 is a decaying exponential:

P(t)=exp(−kt)=exp(−t/τ_{D}) (7)

Here, P(0)=1, corresponding to no decay at t=0, and τ_{D}=1/k is the donor lifetime.

The separate effects of energy transfer, quenching, and other decay mechanisms on luminescence lifetime can be identified and corrected for in part by identifying their separate effects on the rate constant k of Equations 6 and 7. Generally, the rate constant k Aim is a sum of rate constants for each mechanism that leads to decay of the excited state. Thus, for a fluorescent luminophore, the general rate constant k can be expressed as a sum of rate constants for fluorescence (k_{f}), dynamic quenching (k_{d}), other deactivation (kₒ), and energy transfer kₑ, among others. Fluorescence is used here to refer generally to emission of a photon, and may include one or both of fluorescence and phosphorescence, depending on the luminophore. Other deactivation is used here as a catchall for all other forms of nonradiative decay, including but not limited to thermal deactivation.

Static quenching also may be modeled using a rate constant. However, the rate constant for static quenching typically is so large that luminophores bound to static quenchers decay only via static quenching, rendering the luminophores nonluminescent. Thus, static quenching is modeled instead using mole fractions.

In principle, these rate constants may differ for each of D_{f}, D_{b}, A_{f}, and A_{b}. Thus, an energy transfer system may be described using rate constants for each of these species, as well as bound fractions of donor relative to acceptor, and donor and acceptor relative to quencher. Formal rate constants for fluorescence, internal conversion, dynamic quenching, and energy transfer are shown in the following table:

| | D_{f} | D_{b} | A_{f} | A_{b} |
|---|---|---|---|---|
| Fluorescence | k_{fdf} | k_{fdb} | k_{faf} | k_{fab} |
| Other deactivation | k_{odf} | k_{odb} | k_{oaf} | k_{oab} |
| Dynamic quenching | k_{qdf} | k_{qdb} | k_{qaf} | k_{qab} |
| Energy transfer | | kₑ | | |

Generally, these rate constants will vary with the specific donor and acceptor, and with different sample and assays conditions. The subscripts “f” and “b” on these rate constants may be dropped if the corresponding rate constants are the same for free and bound states; for example, if k_{odf}=k_{odb}, then both may be labeled k_{od}. The fraction of donor that is bound by acceptor is x. The fractions of free and bound donor and free and bound acceptor that are bound by static quencher (and rendered nonfluorescent) are f_{qdf}, f_{qdb}, f_{qaf}, and f_{qab}, respectively.

The methods provided by the invention may be used in time-resolved luminescence assays, including time-resolved time-domain assays and time-resolved frequency-domain assays. In time-domain assays, donors typically are excited by a pulse of light that is short relative to the apparent lifetimes of each species except A_{f}. The “per molecule” time evolution of the fluorescence of each species following excitation may be described using the following equations, for times that are long relative to the lifetime of A_{f}:

$$\begin{matrix}{F}_{Df} ( t ) = ( 1 - {f}_{qdf} ) ( {k}_{fd} ) exp ( - t / {τ}_{Df} ) & \text{(8a)} \\ {F}_{Db} ( t ) = ( 1 - {f}_{qdb} ) ( {k}_{fb} ) exp ( - t / {τ}_{Db} ) & \text{(8b)} \\ {F}_{Af} ( t ) \approx 0 & \text{(8c)} \\ {F}_{Ab} ( t ) = ( 1 - {f}_{qab} ) ( {k}_{e} ) [ {k}_{fa} / ( {k}_{fa} + {k}_{oa} + {k}_{qab} ) ] exp ( - t / {τ}_{Ab} ) & \text{(8d)}\end{matrix}$$

Here, τ_{Df}=1/(k_{qdf}+kₒ+k_{fd}) is the lifetime of D_{f}, τ_{Db}=1/(k_{qdb}+k_{od}+k_{fd}+kₑ) is the lifetime of D_{b}, and τ_{Ab} is the lifetime of A_{b}. If τ_{Db} is much larger than the lag between energy transfer from D_{b} to A_{b} and subsequent emission from A_{b}, then τ_{Ab} may to good approximation be set equal to τ_{Db}. The fluorescence of free acceptor is about zero, because the equations apply for t>>τ_{Af} and/or because the free acceptor is not significantly excited by light used to excite the donor. In Equation 8, the exponential time dependence from Equation 7 is multiplied by a first term that reduces the fluorescence to account for the fraction of luminophores quenched by quencher, and by a second term that reduces the fluorescence to account for nonradiative decay mechanisms.

Equation 8 is applicable for t>>τ_{Af}, where τ_{Db}>>τ_{Af}. These conditions were chosen because they simplify the analysis and because they correspond to common experimental conditions. However, these conditions may be relaxed within the scope of the invention, and equations analogous to Equation 8 may be derived to describe these relaxed conditions, such as where τ_{Db} is comparable to τ_{Af}.

In a RET experiment, donor and acceptor emission intensities may be recorded separately in different channels corresponding to different wavelengths. The observed decay of fluorescence in each channel is a weighted sum of the observed decays for the free and bound species:

F_{D}(t;λ_{D})=(1−x)F_{Df}(t)+xF_{Db}(t) (9a)

F_{A}(t;λ_{A})=xF_{Ab}(t) (9b)

Here, λ_{D} and λ_{A} denote the range of wavelengths over which luminescence is detected for donor and acceptor, respectively. Equation 9 may be generalized to account for spectral crosstalk between D and A channels; for example, if 1% of donor luminescence is detected in the acceptor channel, F_{A}(t) may be corrected by the transformation F_{A}(t)→F_{A}(t)−0.01F_{D}(t).

Equations 8 and 9 may be made more accurate by including additional coefficients and dependencies and by relaxing simplifying assumptions. For example, an instrumental gain coefficient can be used to quantify detection efficiencies and to account for differences in detection efficiency of donor and acceptor luminescence. The simplifications and assumptions addressed by these and other modifications do not affect the qualitative conclusions described herein.

Equations 8 and 9 (or their more detailed analogs) may be used to develop tables of expected effects of quenching and other conditions on lifetimes and intensities in RET and other luminescence assays. These tables in turn may be used to identify “false positive” or “false negative” experimental results, where the positive or negative result is at least partially due to quenching. Here, table refers to any representation showing how lifetimes and/or intensities are affected by quenching and other effects, and not merely to a physical representation of such relationships, such as an arrangement of ordered rows or columns.

The following example illustrates how the method may be used to develop such a table and to identify and correct for quenching effects in a time-domain RET assay. The experimental system is characterized by various parameters, which describe donor, acceptor, quencher, apparatus, and detection protocol. Generally, these parameters may be measured and/or estimated.

Donor, acceptor, and quencher were characterized by rate constants and bound fractions. Here, parameters were roughly characteristic of lanthanide assay systems. Lifetimes and intensities were assumed to be affected by fluorescence, other deactivation, energy transfer, and dynamic and static quenching. Donor/acceptor binding were assumed to range between a minimum of xₘᵢₙ=0.1% donor bound by acceptor to a maximum of xₘₐₓ=50% donor bound by acceptor. Fluorescence, other deactivation, and energy transfer were characterized by the following rate constants:

| Rate Constants (μs⁻¹) | D_{f} | D_{b} | A_{b} |
|---|---|---|---|
| Fluorescence (k_{f}) | 0.002 | 0.002 | 250 |
| Other Deactivation (kₒ) | 0.0001 | 0.0001 | 50 |
| Energy Transfer (kₑ) | | 0.008 | 0 |

Dynamic quenching was characterized by rate constants (k_{q}) that ranged between 0 μs⁻¹ and 0.002 μs⁻¹. Static quenching was characterized by bound fractions f_{qdf}, f_{qdb}, f_{qaf}, and f_{qab} that ranged between 0% and 50%.

Apparatus and detection protocol were characterized by a detection efficiency and a crosstalk. Here, detection efficiency was assumed to be 100% for donor and acceptor luminescence, and crosstalk between donor and acceptor detection channels was assumed to be 0% for donor detection in the acceptor channel (relative to donor detection in the donor channel) and 0% for acceptor detection in the donor channel (relative to acceptor detection in the acceptor channel).

The effects of donor and acceptor binding and dynamic and static quenching may be characterized by evaluating Equations 8 and 9 for the parameters listed above using a computerized spreadsheet. Results may be determined for specific times by evaluating the equations at the specific times. Results may be determined for ranges of times (corresponding to experimental time windows) by integrating the equations over the ranges of times.

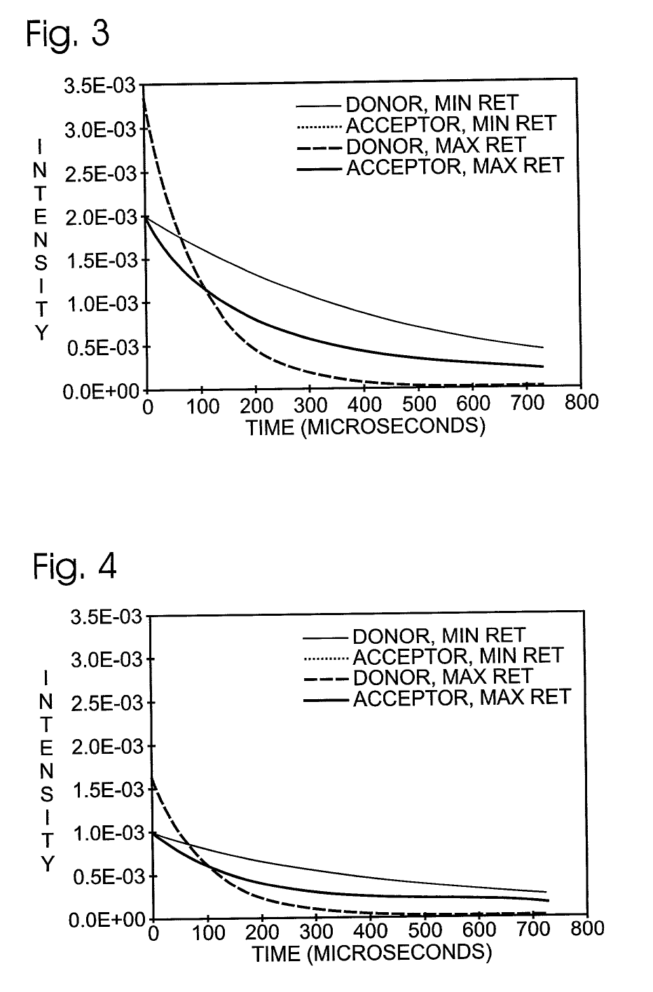

FIG. 3 shows luminescence intensities for acceptor and donor as functions of time and energy transfer. The associated lifetimes are 476 microseconds for D_{f} and 99 microseconds for D_{b} and A_{b}. Minimum and maximum RET correspond to 0.1% and 50% binding of acceptor to donor, simulating the modulation of energy transfer in a binding assay. The donor curve is a sum of emissions from free and bound donor. The acceptor curve arises only from bound acceptor, because free acceptor generally is not appreciably excited directly, and because emissions from free acceptor already have decayed on the time scale shown in the figure. The acceptor curve under minimum energy-transfer conditions is invisible because it is so low that it essentially lies on the time axis.

FIG. 3 also shows the effects of energy transfer: a decrease in donor intensity, an increase in acceptor intensity, a more rapid decay of acceptor than free donor, and a more rapid (and bi-exponential) decay of the donor signal due to the appearance of a component from the more rapidly decaying bound donor.

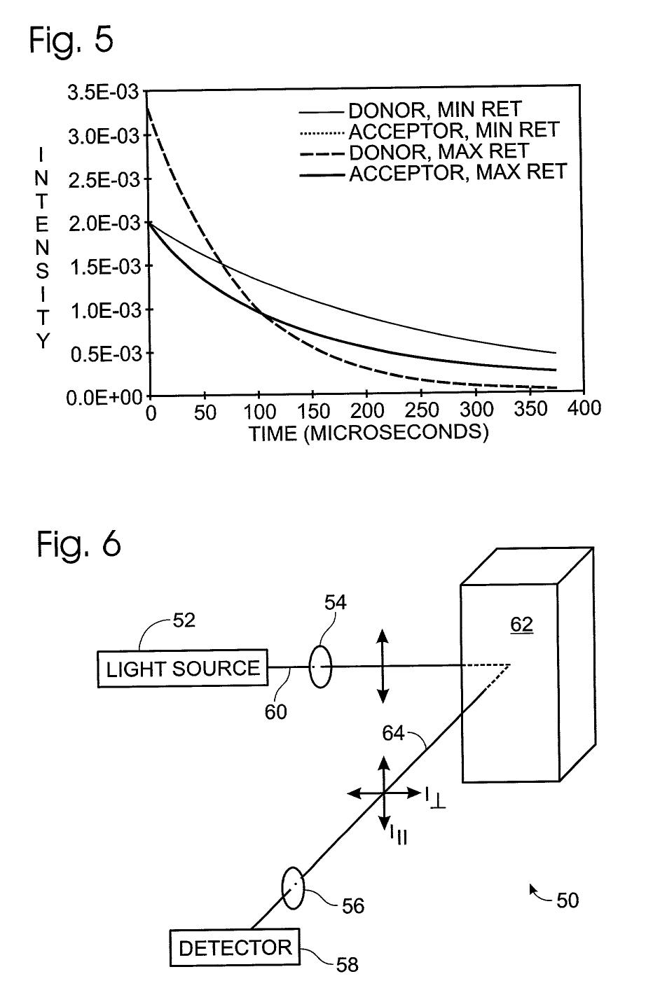

FIGS. 4 and 5 show the effects of static and dynamic quenching on the energy transfer system of FIG. 3. It is possible to create many examples with different types and amounts of quenching on the various species in the assay. For simplicity, and without limitation, these figures show only cases in which there is static or dynamic quenching of the free and bound donor.

FIG. 4 shows luminescence intensities for acceptor and donor as functions of time, energy transfer, and static quenching. Here, 50% of the free and bound donor are statically quenched. Static quenching reduces all emissions, starting at t=0. However, static quenching does not affect lifetimes of individual species, so that the rate of decay of the signals is not appreciably altered.

FIG. 5 shows luminescence intensities for acceptor and donor as functions of time, energy transfer, and dynamic quenching. Here, free and bound donor are dynamically quenched, with a rate constant of 0.002/microsecond, giving about 50% quenching of free donor and somewhat less quenching of bound donor. In this case, emissions are unaffected at t=0 but decay more rapidly because lifetimes of the individual species have been reduced (to 244 microseconds for free donor, and 83 microseconds for bound donor and bound acceptor).

Collecting lifetime information is a valuable adjunct to collecting intensities integrated over a fixed time window. In particular, intensities and lifetimes may be analyzed together to distinguish decreases in binding-derived energy transfer from static and dynamic quenching. For example, based on a comparison of results from FIGS. 3, 4, and 5, reduced lifetimes are diagnostic for dynamic quenching.

The following table shows how changes in donor and acceptor binding and dynamic and static quenching differentially affect species lifetimes and species intensities:

| | |
|---|---|
| Effects on | Effects on |
| Species Lifetimes | Species Intensities |

| Condition . . . | D_{f} | D_{b} | A_{b} | A_{b}/D_{f} | D_{f} | D_{b} | A_{b} | A_{b}/D_{f} |
|---|---|---|---|---|---|---|---|---|
| Increased D:A | — | — | — | — | ↓ | ↑ | ↑ | ↑ |
| Binding | | | | | | | | |
| Decreased D:A | — | — | — | — | ↑ | ↓ | ↓ | ↓ |
| Binding | | | | | | | | |
| Dynamic Quenching | ↓ | — | — | ↑ | ↓ | — | — | ↑ |
| of D_{f} | | | | | | | | |
| Dynamic Quenching | ↓ | ↓ | ↓ | — | ↓ | ↓ | ↓ | — |
| of D_{f} and D_{b} equally | | | | | | | | |
| Dynamic Quenching | — | — | ↓ | ↓ | — | — | ↓ | ↓ |
| of A_{b} | | | | | | | | |
| Static Quenching | — | — | — | — | ↓ | — | — | ↑ |
| of D_{f} | | | | | | | | |
| Static Quenching | — | — | — | — | ↓ | ↓ | ↓ | — |
| of D_{f} and D_{b} equally | | | | | | | | |
| Static Quenching | — | — | — | — | — | — | ↓ | ↓ |
| of A_{b} | | | | | | | | |

The table generally applies to a broad range of conditions, even if entries were derived in some cases using specific parameters. “Changes” refers generally to relative changes, such as an incremental increase or decrease in D:A binding relative to an arbitrary initial value. Changes also may refer more specifically to changes relative to a blank or control that for example does not include a target analyte and/or a quencher. “Species” refers to D_{f}, D_{b}, A_{f}, and A_{b} considered separately, rather than in combination. Species lifetimes are intensive quantities, and species intensities are extensive quantities.

The first two rows of the table show how changes in the amount of tl donor: acceptor binding affect species lifetimes and species intensities. Changes in binding do not affect species lifetimes, although they do affect mean (weighted average of free and bound) lifetimes. In contrast, changes in binding do affect species intensities. Specifically, increases in binding decrease intensities from free donor and increase intensities from bound donor and acceptor. Conversely, decreases in binding increase intensities from free donor and decrease intensities from bound donor and acceptor.

The third through fifth rows of the table show how dynamic quenching of D_{f}, D_{f} and D_{b}, or A_{b} affects species lifetimes and species intensities. Generally, dynamic quenching of a species always decreases the lifetime and intensity of that species. Thus, because changes in donor: acceptor binding generally do not affect species lifetimes, decreases in species lifetimes are diagnostic for dynamic quenching.

The sixth through eighth rows of the table show how static quenching of D_{f}, D_{f} and D_{b}, or A_{b} affects species lifetimes and species intensities. Generally, static quenching does not affect species lifetimes, but decreases species intensities. Static quenching resembles a decrease in donor: acceptor binding, except that a decrease in donor: acceptor binding is accompanied by a decrease in intensity of bound donor and static quenching is not.

The invention may be implemented to carry out a screening protocol in which individual qualitative tests or assays are performed on a number of samples. Screening protocols typically result in a relatively small number of true positives, and additionally, some number of false positives and false negatives. If the number of false positives and/or false negatives are too high, then the utility of the screening protocol may be significantly undermined. Adjusting the sensitivity of the test to decrease the number of false positives typically will cause some increase in the number of false negatives. Conversely, adjusting the sensitivity of the test to decrease the number of false negatives often will cause an increase in the number of false positives. The table showing relationships of lifetime and intensity changes in relation to quenching effects may be used to program the instrument so that the sensitivity of an assay in a screening protocol is optimally set to minimize false positives and false negatives, thus improving the overall efficiency of the procedure.

Apparent quenching can arise due to optical properties of a sample, including optical density and turbidity. A common example is “color quenching”, which is a reduction of measured luminescence intensities (without a change in lifetimes) by Beer-Lambert absorption of excitation and/or emission light by chromophores present in the assay solution at appreciable optical densities. Color quenching is common in screens for new pharmaceuticals, where library compounds may have significant extinction coefficients at the excitation and/or emission wavelengths of the labels. Color quenching occurs separately from the molecular photophysics of the donors and acceptors. A ratio of acceptor-to-donor emission can correct for absorption at donor excitation wavelengths, because such absorption will reduce all luminescence intensities to the same extent. This ratio also can correct for equal absorption (optical density) at donor and acceptor emission wavelengths. However, this (or another) ratio will not easily correct for unequal absorption (optical density) at donor and acceptor emission wavelengths, which if uncorrected may mimic changes in binding. In this case, lifetime measurements may be useful for correcting for color quenching; see PCT Patent Application Ser. No. Leo PCT/US99/01656, which is incorporated herein by reference.

Time-resolved RET assays typically are detected by monitoring integrated intensities using a single time window for donor and a single time window for acceptor. To gather the information discussed herein, lifetimes and intensities can be determined in time-domain measurements by collecting data in multiple time windows, preferably more than two for each wavelength monitored. Alternatively, measurements can be done in the frequency domain, by exciting with amplitude-modulated light and measuring the phase and modulation of the emissions as a function of the frequency of excitation modulation. The frequency-domain results can be couched in terms of effects on directly measured phase angles and modulation (and perhaps unmodulated intensity) instead of derived lifetime and intensity. The analytical treatment of frequency-domain results differs in detail but not in spirit from the analytical treatment of time-domain results presented here, embodying the same photophysics. Substantially the same information is contained in ideal time-domain and frequency-domain results, although practical instrumental factors may render them of different utility.

In summary, combined measurements of lifetimes and intensities may be used to identify and correct for various forms of quenching, so that quenching can be distinguished from increases or decreases in the extent of donor/acceptor binding.

### 3. Description of Apparatus

FIG. 6 shows an apparatus 50 for detecting light (including polarized light) leaving a sample. Apparatus 50 includes a light source 52, an excitation polarizer 54, an emission polarizer 56, and a detector 58. Light 60 produced by light source 52 is directed through excitation polarizer 54, which passes polarized excitation light (indicated by vertical arrow). Polarized excitation light is directed onto a sample 62, which emits light 64 in response. The emitted light may be either some fraction of the incident light or luminescence. Emitted light 64 is directed through emission polarizer 56, which may have components oriented parallel (∥; indicated by vertical arrow) or perpendicular (⊥; indicated by horizontal arrow) to the polarization of excitation light 60. Depending on its orientation, emission polarizer 56 passes parallel (I_{∥}) or perpendicular (I_{⊥}) components of emission light 64 for detection by detector 58.

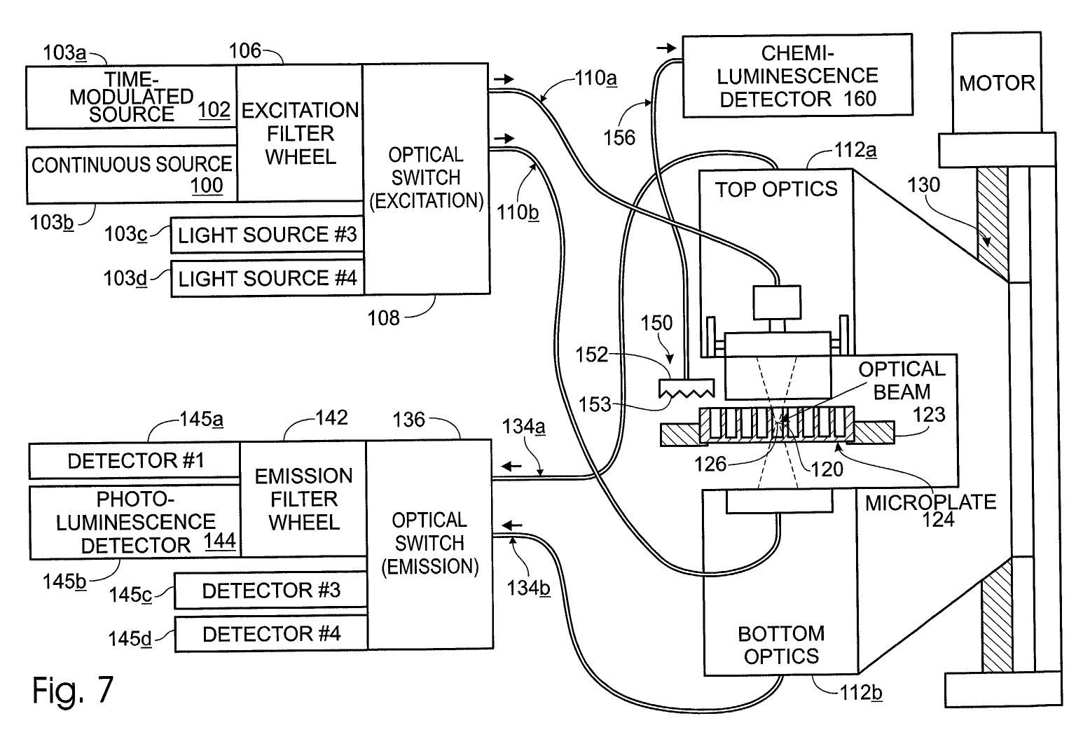

FIGS. 7-10 show an alternative apparatus 90 for detecting light emitted by an analyte in a composition. Apparatus 90 includes (1) a stage for supporting the composition, (2) one or more light sources for delivering light to a composition, (3) one or more detectors for receiving light transmitted from the composition and converting it to a signal, (4) first and second optical relay structures for relaying light between the light source, composition, and detector, and (5) a processor for analyzing the signal from the detector. All or only a subset of these components may be used in any given application.

Apparatus 90 may be used for a variety of assays, including but not limited to the assays described above. Components of the optical system may be chosen to optimize sensitivity and dynamic range for each assay supported by the apparatus. Toward this end, optical components with low intrinsic luminescence are preferred. In addition, some components may be shared by different modes, whereas other components may be unique to a particular mode. For example, in apparatus 90, absorbance, scattering, photoluminescence intensity and steady-state photoluminescence polarization modes share a light source; time-resolved absorbance and luminescence modes use their own light source; and chemiluminescence modes do not use a light source. Similarly, photoluminescence and chemiluminescence modes use different detectors.

The remainder of this section is divided into four subsections: (A) incident light-based optical system, (B) chemiluminescence optical system, (C) housing, and (D) frequency-domain detection system.

A. Incident Light-Based Optical System

FIGS. 7-9 show the incident light-based optical system of apparatus 90. As configured here, apparatus 90 includes a continuous light source 100 and a time-modulated light source 102. Apparatus 90 includes light source slots 103a-d for four light sources, although other numbers of light source slots and light sources also could be provided. Light source slots 103a-d function as housings that may surround at least a portion of each light source, providing some protection from radiation and explosion. The direction of light transmission through the incident light-based optical system is indicated by arrows.

Continuous source 100 provides light for absorbance, scattering, photoluminescence intensity, and steady-state photoluminescence polarization assays. Continuous light source 100 may include arc lamps, incandescent lamps, fluorescent lamps, electroluminescent devices, lasers, laser diodes, and light-emitting diodes (LEDs), among others. A preferred continuous source is a high-intensity, high color temperature xenon arc lamp, such as a Model LX175F CERMAX xenon lamp from ILC Technology, Inc. Color temperature is the absolute temperature in Kelvin at which a blackbody radiator must be operated to have a chromaticity equal to that of the light source. A high color temperature lamp produces more light than a low color temperature lamp, and it may have a maximum output shifted toward or into visible wavelengths and ultraviolet wavelengths where many luminophores absorb. The preferred continuous source has a color temperature of 5600 Kelvin, greatly exceeding the color temperature of about 3000 Kelvin for a tungsten filament source. The preferred source provides more light per unit time than flash sources, averaged over the flash source duty cycle, increasing sensitivity and reducing read times. Apparatus 90 may include a modulator mechanism configured to vary the intensity of light incident on the composition without varying the intensity of light produced by the light source.

Time-modulated source 102 provides light for time-resolved absorbance and/or photoluminescence assays, such as photoluminescence lifetime and time-resolved photoluminescence polarization assays. A preferred time-modulated source is a xenon flash lamp, such as a Model FX-1160 xenon flash lamp from EG&G Electro-Optics. The preferred source produces a “flash” of light for a brief interval before signal detection and is especially well suited for time-domain measurements. Other time-modulated sources include pulsed lasers, electronically modulated lasers and LEDs, and continuous lamps and other sources whose intensity can be modulated extrinsically using a Pockels cell, Kerr cell, or other mechanism. Such other mechanisms may include an amplitude modulator such as a chopper as described in U.S. Provisional Patent Application No. 60/094,276, which is incorporated herein by reference. Extrinsically modulated continuous light sources are especially well suited for frequency-domain measurements.

In apparatus 90, continuous source 100 and time-modulated source 102 produce multichromatic, unpolarized, and incoherent light. Continuous source 100 produces substantially continuous illumination, whereas time-modulated source 102 produces time-modulated illumination. Light from these light sources may be delivered to the sample without modification, or it may be filtered to alter its intensity, spectrum, polarization, or other properties.

Light produced by the light sources follows an excitation optical path to an examination site or measurement region. Such light may pass through one or more “spectral filters, ” which generally comprise any mechanism for altering the spectrum of light that is delivered to the sample. Spectrum refers to the wavelength composition of light. A spectral filter may be used to convert white or multichromatic light, which includes light of many colors, into red, blue, green, or other substantially monochromatic light, which includes light of one or only a few colors. In apparatus 90, spectrum is altered by an excitation interference filter 104, which preferentially transmits light of preselected wavelengths and preferentially absorbs light of other wavelengths. For convenience, excitation interference filters 104 may be housed in an excitation filter wheel 106, which allows the spectrum of excitation light to be changed by rotating a preselected filter into the optical path. Spectral filters also may separate light spatially by wavelength. Examples include gratings, monochromators, and prisms.

Spectral filters are not required for monochromatic (“single color”) light sources, such as certain lasers, which output light of only a single wavelength. Therefore, excitation filter wheel 106 may be mounted in the optical path of some light source slots 103a,b, but not other light source slots 103c,d. Alternatively, the filter wheel may include a blank station that does not affect light passage.

Light next passes through an excitation optical shuttle (or switch) 108, which positions an excitation fiber optic cable 110a,b in front of the appropriate light source to deliver light to top or bottom optics heads 112a,b, respectively. Light is transmitted through a fiber optic cable much like water is transmitted through a garden hose. Fiber optic cables can be used easily to turn light around corners and to route light around opaque components of the apparatus. Moreover, fiber optic cables give the light a more uniform intensity profile. A preferred fiber optic cable is a fused silicon bundle, which has low autoluminescence. Despite these advantages, light also can be delivered to the optics heads using other mechanisms, such as mirrors.

Light arriving at the optics head may pass through one or more excitation “polarization filters, ” which generally comprise any mechanism for altering the polarization of light. Excitation polarization filters may be included with the top and/or bottom optics head. In apparatus 90, polarization is altered by excitation polarizers 114, which are included only with top optics head 112a for top reading; however, such polarizers also can be included with bottom optics head 112b for bottom reading. Excitation polarization filters 114 may include an s-polarizer S that passes only s-polarized light, a p-polarizer P that passes only p-polarized light, and a blank O that passes substantially all light. Excitation polarizers 114 also may include a standard or ferro-electric liquid crystal display (LCD) polarization switching system. Such a system may be faster than a mechanical switcher. Excitation polarizers 114 also may include a continuous mode LCD polarization rotator with synchronous detection to increase the signal-to-noise ratio in polarization assays. Excitation polarizers 114 may be incorporated as an inherent component in some light sources, such as certain lasers, that intrinsically produce polarized light.

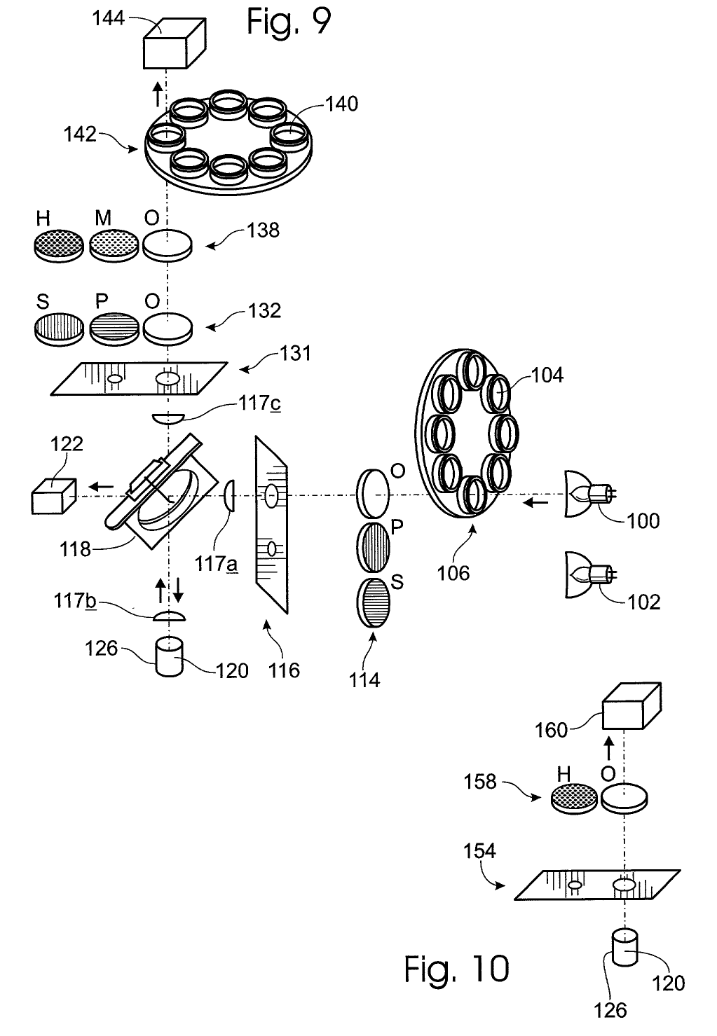

Light at one or both optics heads also may pass through an excitation “confocal optics element, ” which generally comprises any mechanism for focusing light into a “sensed volume.” In apparatus 90, the confocal optics element includes a set of lenses 117a-c and an excitation aperture 116 placed in an image plane conjugate to the sensed volume, as shown in FIG. 9. Aperture 116 may be implemented directly, as an aperture, or indirectly, as the end of a fiber optic cable. Preferred apertures have diameters of 1 mm and 1.5 mm. Lenses 117a,b project an image of aperture 116 onto the sample, so that only a preselected or sensed volume of the sample is illuminated. The area of illumination will have a diameter corresponding to the diameter of the excitation aperture.

Light traveling through the optics heads is reflected and transmitted through a beamsplitter 118, which delivers reflected light to a composition 120 and transmitted light to a light monitor 122. Reflected and transmitted light both pass through lens 117b which is operatively positioned between beamsplitter 118 and composition 120.

Beamsplitter 118 is used to direct excitation or incident light toward the sample and light monitor, and to direct light leaving the sample toward the detector. The beamsplitter is changeable, so that it may be optimized for different assay modes or compositions. If a large number or variety of photoactive molecules are to be studied, the beamsplitter must be able to accommodate light of many wavelengths; in this case, a “50:50” beamsplitter that reflects half and transmits half of the incident light independent of wavelength is optimal. Such a beamsplitter can be used with many types of molecules, while still delivering considerable excitation light onto the composition, and while still transmitting considerable light leaving the sample to the detector. If one or a few related photoactive molecules are to be studied, the beamsplitter needs only to be able to accommodate light at a limited number of wavelengths; in this case, a “dichroic” or “multichroic” beamsplitter is optimal. Such a beamsplitter can be designed with cutoff wavelengths for the appropriate sets of molecules and will reflect most or substantially all of the excitation and background light, while transmitting most or substantially all of the emission light in the case of luminescence. This is possible because the beamsplitter may have a reflectivity and transmissivity that varies with wavelength.

Light monitor 122 is used to correct for fluctuations in the intensity of light provided by the light sources. Such corrections may be performed by reporting detected intensities as a ratio over corresponding times of the luminescence intensity measured by the detector to the excitation light intensity measured by the light monitor. The light monitor also can be programmed to alert the user if the light source fails. A preferred light monitor is a silicon photodiode with a quartz window for low autoluminescence.

The composition (or sample) may be held in a sample holder supported by a stage 123. The composition can include compounds, mixtures, surfaces, solutions, emulsions, suspensions, cell cultures, fermentation cultures, cells, tissues, secretions, and/or derivatives and/or extracts thereof. Analysis of the composition may involve measuring the presence, concentration, or physical properties (including interactions) of a photoactive analyte in such a composition. Composition may refer to the contents of a single microplate well, or several microplate wells, depending on the assay. In some embodiments, such as a portable apparatus, the stage may be intrinsic to the instrument.

The sample holder can include microplates, biochips, or any array of samples in a known format. In apparatus 90, the preferred sample holder is a microplate 124, which includes a plurality of microplate wells 126 for holding compositions. Microplates are typically substantially rectangular holders that include a plurality of sample wells for holding a corresponding plurality of samples. These sample wells are normally cylindrical in shape although rectangular or other shaped wells are sometimes used. The sample wells are typically disposed in regular arrays. The “standard” microplate includes 96 cylindrical sample wells disposed in a 8×12 rectangular array on 9 millimeter centers.

The sensed volume typically has an hourglass shape, with a cone angle of about 25° and a minimum diameter ranging between 0.1 mm and 2.0 mm. For 96-well and 384-well microplates, a preferred minimum diameter is about 1.5 mm. For 1536-well microplates, a preferred minimum diameter is about 1.0 mm. The size and shape of the sample holder may be matched to the size and shape of the sensed volume, as described in PCT Patent Application Ser. No. PCT/US99/08410, which is incorporated herein by reference.

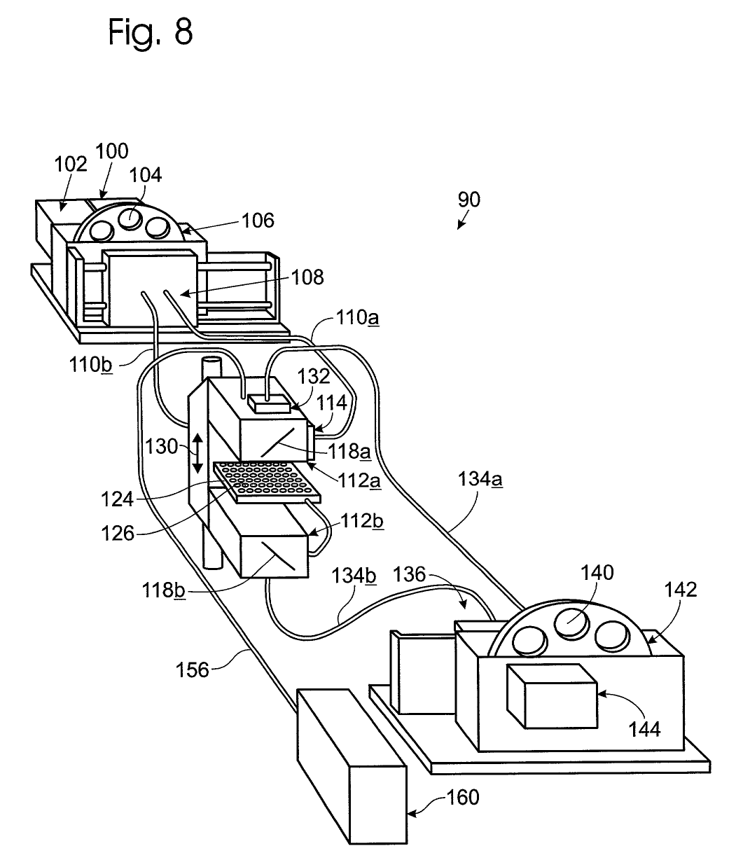

The position of the sensed volume can be moved precisely within the composition to optimize the signal-to-noise and signal-to-background ratios. For example, the sensed volume may be moved away from walls in the sample holder to optimize signal-to-noise and signal-to-background ratios, reducing spurious signals that might arise from luminophores bound to the walls and thereby immobilized. In apparatus 90, position in the X,Y-plane perpendicular to the optical path is controlled by moving the stage supporting the composition, whereas position along the Z-axis parallel to the optical path is controlled by moving the optics heads using a Z-axis adjustment mechanism 130, as shown in FIGS. 7 and 8. However, any mechanism for bringing the sensed volume into register or alignment with the appropriate portion of the composition also may be employed.

The combination of top and bottom optics permits assays to combine: (1) top illumination and top detection, or (2) top illumination and bottom detection, or (3) bottom illumination and top detection, or (4) bottom illumination and bottom detection. Same-side illumination and detection, (1) and (4), is referred to as “epi” and is preferred for photoluminescence and scattering assays. Opposite-side illumination and detection, (2) and (3), is referred to as “trans” and has been used in the past for absorbance assays. In apparatus 90, epi modes are supported, so the excitation and emission light travel the same path in the optics head, albeit in opposite or anti-parallel directions. However, trans modes also can be used with additional sensors, as described below. In apparatus 90, top and bottom optics heads move together and share a common focal plane. However, in other embodiments, top and bottom optics heads may move independently, so that each can focus independently on the same or different sample planes.

Generally, top optics can be used with any sample holder having an open top, whereas bottom optics can be used only with sample holders having optically transparent bottoms, such as glass or thin plastic bottoms. Clear bottom sample holders are particularly suited for measurements involving analytes that accumulate on the bottom of the holder.

Light is transmitted by the composition in multiple directions. A portion of the transmitted light will follow an emission pathway to a detector. Transmitted light passes through lens 117c and may pass through an emission aperture 131 and/or an emission polarizer 132. In apparatus 90, the emission aperture is placed in an image plane conjugate to the sensed volume and transmits light substantially exclusively from this sensed volume. In apparatus 90, the emission apertures in the top and bottom optical AO systems are the same size as the associated excitation apertures, although other sizes also may be used. The emission polarizers are included only with top optics head 112a. The emission aperture and emission polarizer are substantially similar to their excitation counterparts. Emission polarizer 132 may be included in detectors that intrinsically detect the polarization of light.

Excitation polarizers 114 and emission polarizers 132 may be used together in nonpolarization assays to reject certain background signals. Luminescence from the sample holder and from luminescent molecules adhered to the sample holder is expected to be polarized, because the rotational mobility of these molecules should be hindered. Such polarized background signals can be eliminated by “crossing” the excitation and emission polarizers, that is, setting the angle between their transmission axes at 90°. As described above, such polarized background signals also can be reduced by moving the sensed volume away from walls of the sample holder. To increase signal level, beamsplitter 118 should be optimized for reflection of one polarization and transmission of the other polarization. This method will work best where the luminescent molecules of interest emit relatively unpolarized light, as will be true for small luminescent molecules in solution.

Transmitted light next passes through an emission fiber optic cable 134a,b to an emission optical shuttle (or switch) 136. This shuttle positions the appropriate emission fiber optic cable in front of the appropriate detector. In apparatus 90, these components are substantially similar to their excitation counterparts, although other mechanisms also could be employed.

Light exiting the fiber optic cable next may pass through one or more emission “intensity filters, ” which generally comprise any mechanism for reducing the intensity of light. Intensity refers to the amount of light per unit area per unit time. In apparatus 90, intensity is altered by emission neutral density filters 138, which absorb light substantially independent of its wavelength, dissipating the absorbed energy as heat. Emission neutral density filters 138 may include a high-density filter H that absorbs most incident light, a medium-density filter M that absorbs somewhat less incident light, and a blank O that absorbs substantially no incident light. These filters may be changed manually, or they may be changed automatically, for example, by using a filter wheel. Intensity filters also may divert a portion of the light away from the sample without absorption. Examples include beam splifters, which transmit some light along one path and reflect other light along another path, and Pockels cells, which deflect light along different paths through diffraction. Examples also include hot mirrors or windows that transmit light of some wavelengths and absorb light of other wavelengths.

Light next may pass through an emission interference filter 140, which may be housed in an emission filter wheel 142. In apparatus 90, these components are substantially similar to their excitation counterparts, although other mechanisms also could be employed. Emission interference filters block stray excitation light, which may enter the emission path through various mechanisms, including reflection and scattering. If unblocked, such stray excitation light could be detected and misidentified as photoluminescence, decreasing the signal-to-background ratio. Emission interference filters can separate photoluminescence from excitation light because photoluminescence has longer wavelengths than the associated excitation light. Luminescence typically has wavelengths between 200 and 2000 nanometers.

The relative positions of the spectral, intensity, polarization, and other filters presented in this description may be varied without departing from the spirit of the invention. For example, filters used here in only one optical path, such as intensity filters, also may be used in other optical paths. In addition, filters used here in only top or bottom optics, such as polarization filters, may also be used in the other of top or bottom optics or in both top and bottom optics. The optimal positions and combinations of filters for a particular experiment will depend on the assay mode and the composition, among other factors.

Light last passes to a detector, which is used in absorbance, scattering and photoluminescence assays. In apparatus 90, there is one detector 144, which detects light from all modes. A preferred detector is a photomultiplier tube (PMT). Apparatus 90 includes detector slots 145a-d for four detectors, although other numbers of detector slots and detectors also could be provided.

More generally, detectors comprise any mechanism capable of converting energy from detected light into signals that may be processed by the apparatus, and by the processor in particular. Suitable detectors include photomultiplier tubes, photodiodes, avalanche photodiodes, charge-coupled devices (CCDs), and intensified CCDs, among others. Depending on the detector, light source, and assay mode, such detectors may be used in a variety of detection modes. These detection modes include (1) discrete (e.g., photon-counting) modes, (2) analog (e.g., current-integration) modes, and/or (3) imaging modes, among others, as described in PCT Patent Application Ser. No. PCT/US99/03678.

B. Chemiluminescence Optical System

FIGS. 7, 8, and 10 show the chemiluminescence optical system of apparatus 50. Because chemiluminescence follows a chemical event rather than the absorption of light, the chemiluminescence optical system does not require a light source or other excitation optical components. Instead, the chemiluminescence optical system requires only selected emission optical components. In apparatus 50, a separate lensless chemiluminescence optical system is employed, which is optimized for maximum sensitivity in the detection of chemiluminescence.

Generally, components of the chemiluminescence optical system perform the same functions and are subject to the same caveats and alternatives as their counterparts in the incident light-based optical system. The chemiluminescence optical system also can be used for other assay modes that do not require illumination, such as electrochemiluminescence.

The chemiluminescence optical path begins with a chemiluminescent composition 120 held in a sample holder 126. The composition and sample holder are analogous to those used in photoluminescence assays; however, analysis of the composition involves measuring the intensity of light generated by a chemiluminescence reaction within the composition rather than by light-induced photoluminescence. A familiar example of chemiluminescence is the glow of the firefly.

Chemiluminescence light typically is transmitted from the composition in all directions, although most will be absorbed or reflected by the walls of the sample holder. A portion of the light transmitted through the top of the well is collected using a chemiluminescence head 150, as shown in FIG. 7, and will follow a chemiluminescence optical pathway to a detector. The direction of light transmission through the chemiluminescence optical system is indicated by arrows.

The chemiluminescence head includes a nonconfocal mechanism for transmitting light from a sensed volume within the composition. Detecting from a sensed volume reduces contributions to the chemiluminescence signal resulting from “cross talk, ” which is pickup from neighboring wells. The nonconfocal mechanism includes a chemiluminescence baffle 152, which includes rugosities 153 that absorb or reflect light from other wells. The nonconfocal mechanism also includes a chemiluminescence aperture 154 that further confines detection to a sensed volume.

Light next passes through a chemiluminescence fiber optic cable 156, which may be replaced by any suitable mechanism for directing light from the composition toward the detector. Fiber optic cable 156 is analogous to excitation and emission fiber optic cables 110a,b and 134a,b in the photoluminescence optical system. Fiber optic cable 156 may include a transparent, open-ended lumen that may be filled with fluid. This lumen would allow the fiber optic to be used both to transmit luminescence from a microplate well and to dispense fluids into the microplate well. The effect of such a lumen on the optical properties of the fiber optic could be minimized by employing transparent fluids having optical indices matched to the optical index of the fiber optic.

Light next passes through one or more chemiluminescence intensity filters, which generally comprise any mechanism for reducing the intensity of light. In apparatus 50, intensity is altered by chemiluminescence neutral density filters 158. Light also may pass through other filters, if desired.

Light last passes to a detector, which converts light into signals that may be processed by the apparatus. In apparatus 50, there is one chemiluminescence detector 160. This detector may be selected to optimize detection of blue/green light, which is the type most often produced in chemiluminescence. A preferred detection is a photomultiplier tube, selected for high quantum efficiency and low dark count at chemiluminescence wavelengths (400-500 nanometers).

C. Housing

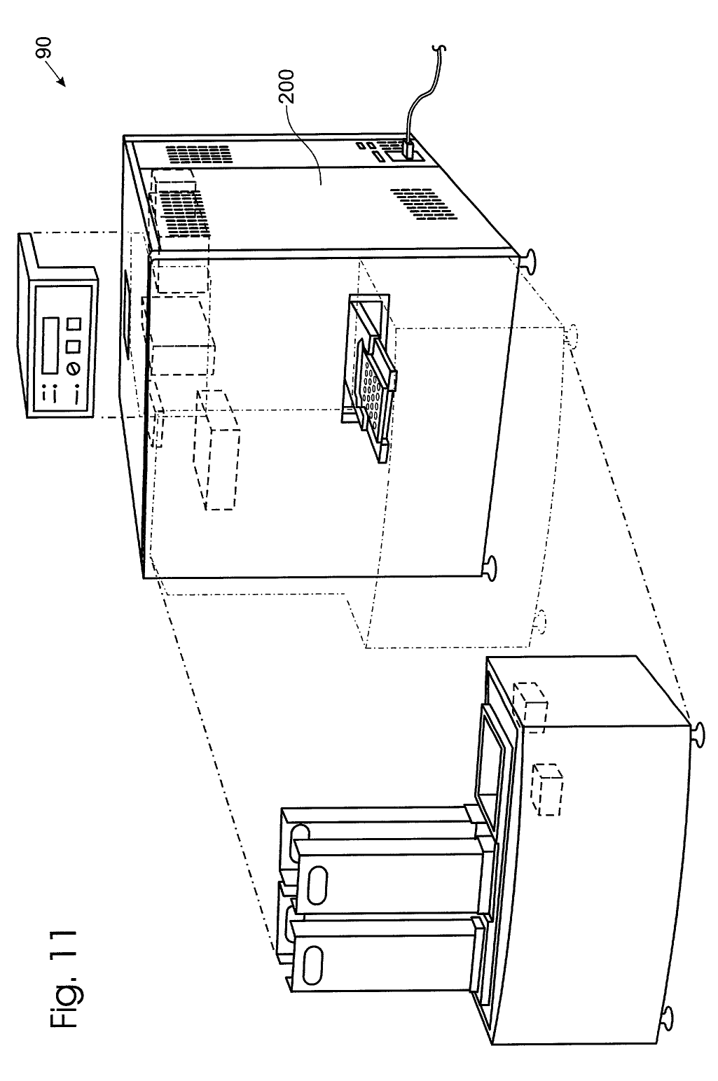

FIG. 11 shows a housing 200 and other accessories for the apparatus of FIGS. 7-10. Housing 200 substantially encloses the apparatus, forming (together with light source slots 103a-d) two protective layers around the continuous high color temperature xenon arc lamp. Housing 200 permits automated sample loading and switching among light sources and detectors, further protecting the operator from the xenon arc lamp and other components of the system. Additional details of an apparatus suitable for implementing features of the invention are shown in U.S. patent application Ser. No. 09/160,533, which is incorporated herein by reference.

D. Frequency-domain Detection System

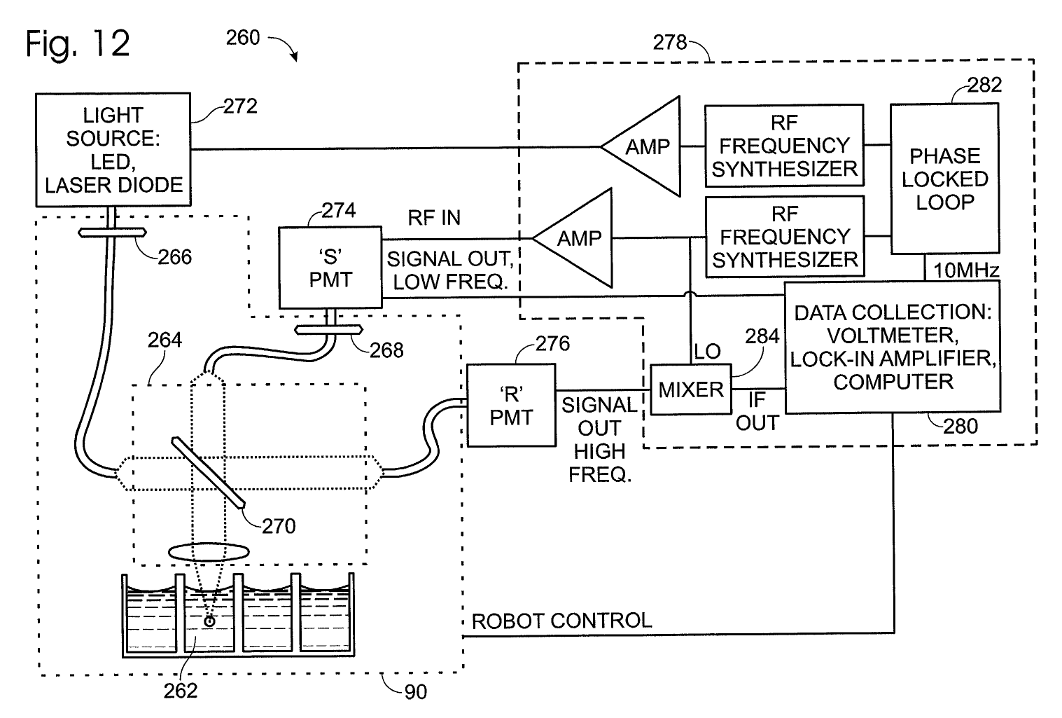

FIG. 12 shows an apparatus 260 for detecting light emitted by an analyte in a composition 262, where the detection and/or processing may be performed in the frequency-domain. Apparatus 260 includes substantial portions of apparatus 90, including its fiber-optic-coupled optics head 264, excitation 266 and emission 268 filters, dichroic beam splitter 270, and mechanisms for sample positioning and focus control. However, apparatus 260 also may include alternative light sources 272, sample (‘S’) detectors 274, reference (‘R’) detectors 276, and detection electronics 278. In FIG. 12, alternative components 272-278 are shown outside apparatus 90, but they readily may be included inside housing 250 of apparatus 90, if desired.

Apparatus 260 may provide incident light in various ways, as described above. For example, analytes absorbing blue light may be excited using a NICHIA-brand bright-blue LED (Model Number NSPB500; Mountville, Pa.). This LED produces broad-spectrum excitation light, so excitation filter 266 may be selected to block the red edge of the spectrum. If analytes are excited using a laser diode, an excitation filter is not necessary.

Apparatus 260 may detect emitted light and convert it to a signal in various ways. This demodulation/deconvolution may be internal to the photodetector, or it may be performed with external electronics or software. For example, emitted light can be detected using sample detector 274, which may be an ISS-brand gain-modulated PMT (Champaign, Ill.). High-frequency emitted light can be frequency down-converted to a low-frequency signal using a technique called heterodyning. The phase and modulation of the low-frequency signal can be determined using a lock-in amplifier 280, such as a STANFORD RESEARCH SYSTEMS brand lock-in amplifier (Model Number SR830; Sunnyvale, Calif.). Lock-in amplifier 280 is phase locked using a phase-locked loop 282 to the modulation frequency of light source 272. To correct for drift in the light source, the output of light source 272 may be monitored using reference detector 276, which may be a HAMAMATSU-brand PMT (Model Number H6780; Bridgewater, N.J.). If reference detector 276 can respond to high-frequency signals, the heterodyning step can be performed using an external mixer 284. The phase and modulation of reference detector 276 also may be captured by lock-in amplifier 280 and used to normalize the signal from sample detector 274.

Apparatus 260 is controlled by a computer or processor. The computer also directs sample handling and data collection. Generally, phase and modulation data are collected at one or more frequencies appropriate for the lifetime of the analyte. In some cases, phase and modulation may be measured at one or a few frequencies and processed by the computer or processor to help reduce detected background.

Although the invention has been disclosed in its preferred forms, the specific embodiments thereof as disclosed and illustrated herein are not to be considered in a limiting sense, because numerous variations are possible. For example, the invention was described primarily in the context of time-domain RET assays, but applies equally to frequency-domain RET assays, as well as other luminescence assays. Applicants regard the subject matter of their invention as including all novel and nonobvious combinations and subcombinations of the various elements, features, functions, and/or properties disclosed herein. No single feature, function, element or property of the disclosed embodiments is essential. The following claims define certain combinations and subcombinations of features, functions, elements, and/or properties that are regarded as novel and nonobvious. Other combinations and subcombinations may be claimed through amendment of the present claims or presentation of new claims in this or a related application. Such claims, whether they are broader, narrower, equal, or different in scope from the original claims, also are regarded as included within the subject matter of applicants' invention.

We claim:

1. A method of performing a luminescence assay, the method comprising the steps of:
performing an assay configured to relate a change in luminescence emission to the presence of a target in a sample;
detecting a change in luminescence emission from the sample; and
identifying at least a portion of the change in luminescence emission which is due to quenching.

2. The method of claim 1, wherein the identifying step includes the step of determining at least a portion of the change in luminescence emission that is due to dynamic quenching.

3. The method of claim 1, wherein the identifying step includes the step of determining at least a portion of the change in luminescence emission that is due to static quenching.

4. The method of claim 1, wherein the performing step includes the step of designing the assay so that a change in luminescence emission may be correlated with RET.

5. The method of claim 1, wherein the performing step includes the step of designing the assay so that a change in luminescence emission may be correlated with time-resolved RET.

6. The method of claim 1 further comprising the step of processing lifetime and intensity measurements to identify a quenching effect.

7. The method of claim 1 further comprising the step of detecting luminescence in multiple time windows.

8. The method of claim 1 further comprising the step of illuminating at least a portion of the sample with pulsed light.

9. The method of claim 1 further comprising the step of analyzing luminescence lifetime and intensity measurements to determine whether a significant portion of detected change in luminescence emission is due to quenching.

10. An apparatus for detecting luminescence, the apparatus comprising:
an instrumentation system capable of detecting changes in luminescence emission from a sample; and
a processor configured to indicate changes in luminescence emission that are due to quenching.

11. The apparatus of claim 10 further comprising a controller that obtains and integrates luminescence intensity and lifetime measurements to determine quenching effects.

12. The apparatus of claim 10 further comprising a controller that processes luminescence detection in multiple time windows.

13. A method of discriminating quenching effects from RET effects in a time-resolved RET assay, the method comprising:
deriving a formula at least partially based on known rate constants relating to luminescence and quenching for each of a donor and an acceptor of a RET pair; and
using the formula to develop a table of expected effects on luminescence lifetimes and intensities in relation to a set of conditions including changes in donor:acceptor binding, and quenching.

14. The method of claim 13, wherein the deriving step results in the following formula:
$$\begin{matrix}{F}_{Df} ( t ) = ( 1 - {f}_{qdf} ) ( {k}_{fd} ) exp ( - t / {τ}_{Df} ) \\ {F}_{Db} ( t ) = ( 1 - {f}_{qdb} ) ( {k}_{fb} ) exp ( - t / {τ}_{Db} ) \\ {F}_{Af} ( t ) \approx 0 \\ {F}_{Ab} ( t ) = ( 1 - {f}_{qab} ) ( {k}_{e} ) [ {k}_{fa} / ( {k}_{fa} + {k}_{oa} + {k}_{qab} ) ] exp ( - t / {τ}_{Ab} )\end{matrix}$$
wherein F_{Df}(t), F_{Db}(t), F_{Af}(t), and F_{Ab}(t) refer to the luminescence of the free donor, bound donor, free acceptor, and bound acceptor, respectively;
wherein f_{qdf}, f_{qdb}, and f_{qab} refer to the fraction of free donor, bound donor, and bound acceptor quenched by static quenchers, respectively;
wherein k_{f}, kₑ, kₒ, and k_{q} are rate constants for luminescence, energy transfer, other deactivation, and dynamic quenching, respectively, for free donor, bound donor, free acceptor, and bound acceptor, as indicated; and
wherein τ_{Df}, τ_{Db}, and τ_{Ab} are lifetimes of free donor, bound donor, and bound acceptor, respectively.

15. The method of claim 13 further comprising the step of performing a time resolved RET assay designed to detect changes in luminescence due to presence of target in a sample.

16. The method of claim 15, wherein the performing step includes the step of detecting changes in luminescence lifetime and intensities of the donor and acceptor.

17. A method of screening a plurality of samples for presence of target, the method comprising:
depositing each sample in a separate sample container;
for each sample, performing a RET assay designed to detect target; and
in each assay, discriminating quenching effects from RET effects due to presence of target.

18. The method of claim 17, wherein the discriminating step includes the step of identifying false positives that are at least partially due to quenching.

19. The method of claim 17 further comprising the step of programming a light detection instrument based on known rate constants relating to luminescence and quenching of a donor and acceptor used in the RET assay.

20. The method of claim 17, wherein the performing step includes the step of detecting changes in luminescence lifetime and intensities of the donor and acceptor.

21. The method of claim 17, wherein the performing step includes the step of exciting a donor and an acceptor by a pulse of light that is short relative to the lifetimes of free donor, bound donor, and bound acceptor, but long relative to the lifetime of free acceptor.

22. The method of claim 17, wherein the performing step includes the step of conducting time-domain measurements by collecting data in multiple time windows to determine changes in luminescence lifetimes and intensities of the donor and the acceptor.

23. The method of claim 17, wherein the performing step includes the step of using frequency-domain measurements to determine changes in luminescence lifetimes and intensifies of the donor and the acceptor.

24. The method of claim 17, wherein the depositing, step includes the step of transferring each sample into a separate microplate well.
